(19) United States
(12) Patent Application Publication
(10) Pub. No.: US 2014/0330153 A1
(43) Pub. Date: Nov. 6, 2014

(54) Selective Point of Care Nanoprobe Breath Analyzer

(71) Applicant: The Research Foundation for the State University of New York, Albany, NY (US)

(72) Inventors: Irene Pelagia Gouma, Port Jefferson, NY (US); Anastasia Rigas, Old Field, NY (US)

(73) Assignee: The Research Foundation for the State University of New York

(21) Appl. No.: 14/334,336

(22) Filed: Jul. 17, 2014

Related U.S. Application Data
(63) Continuation of application No. 11/903,135, filed on Sep. 20, 2007.
Application No. 11/903,135 is a continuation-in-part of application No. 11/351,171, filed on Feb. 9, 2006, now Pat. No. 8,485,983.
Application No. 11/351,171 is a continuation-in-part of application No. 10/419,349, filed on Apr. 21, 2003, now Pat. No. 7,017,389.
(60) Provisional application No. 60/973,066, filed on Sep. 17, 2007.
(60) Provisional application No. 60/845,918, filed on Oct. 26, 2006.
(60) Provisional application No. 60/845,917, filed on Sep. 20, 2006.

(51) Int. Cl. A61B 5/08; G01N 33/497

(52) U.S. Cl. CPC A61B 5/082; G01N 33/497
USPC 600/532

(57) ABSTRACT

Disclosed is a medical diagnostic device for analyzing breath gases and/or skin emissions, including a highly sensitive sensing component for obtaining an emission concentration profile and a database of breath analysis profiles medical condition characteristics.

### PRIORITY

This application is a continuation in part of application Ser. No. 11/351,171, filed with the U.S. Patent and Trademark Office on Feb. 11, 2006, and is a continuation in part of U.S. application Ser. No. 10/419,349, filed Apr. 21, 2003, and claims priority to application Ser. No. 60/374,189, filed with the U.S. Patent and Trademark Office on Apr. 20, 2002, to application Ser. No. 60/845,917, filed with the U.S. Patent and Trademark Office on Sep. 20, 2006, to application Ser. No. 60/845,918, filed with the U.S. Patent and Trademark Office on Sep. 20, 2006 and on Oct. 26, 2006, and to application Ser. No. 60/973,066, filed with the U.S. Patent and Trademark Office on Sep. 17, 2007, the contents of each of which is incorporated herein by reference.

### GOVERNMENT SUPPORT

This invention was made with Government support of Grant No. SGER DMR0224642 awarded by the National Science Foundation. The Government has certain rights in this invention.

### BACKGROUND

1. Field of the Invention

The present invention relates generally to a medical device and protocols to facilitate diagnosis of medical conditions based on breath analysis profiles (E-nose) and, in particular, to use of highly sensitive nanostructured polymorphs of metal oxides in such devices and methods.

2. Background of the Invention

Since the time of Hippocrates, exhaled breath was known to enable non-invasive detection of disease. Exhaled gases, such as ammonia, nitric oxide, aldehydes and ketones have been associated with kidney and liver malfunction, asthma, diabetes, cancer, and ulcers. Other exhaled compounds like ethane, butane, pentane, and carbon disulfide have been connected to abnormal neurological conditions. However, though analysis of body fluids (blood, sputum, urine) for disease diagnoses and monitoring is routine clinical practice, human breath analysis methodologies that exploit the non-invasive nature of such diagnoses are still under-developed and conventional technologies lack specificity, are excessively expensive or lack portability.

Technologies for monitoring exhaled breath require complex and expensive apparatuses that are difficult to calibrate and are often not sufficiently sensitive to provide a high degree of certainty in regard to medical condition diagnosis. To address a concern regarding recalibration of portable, at-home sensors, U.S. Pat. No. 7,220,387 to Flaherty et al., the contents of which are incorporated by reference, discloses a disposable sensor for measuring an analyte in a gaseous sample. A conventional apparatus disclosed by Kearney, D, et al. in Breath Ammonia Measurement in Helicobacter pylori Infection (Digestive Diseases and Sciences, Vol. 47, No. II, pp. 2523-2530, Nov. 2002), provides a fiber optic device placed in the stream of expelled breath that is connected to an optical sensor for detecting whether a patient has H. pylori by measuring for ammonia excreted by the lungs. The fiber optic device of Kearney utilizes a hydrophobic TFE-based membrane to avoid affect of dissolved ions such as ammonia.

However, conventional point of care devices are expensive, and a portable point of care system is required, particularly in regard to assessment of H. pylori and similar infections that colonize the gastroduodenal mucosa discontinuously, causing biopsies to miss infected areas.

Conventional testing is performed utilizing instrumentation that ranges from variations of mass spectrometers to IR detectors that are costly and require a trained operator. Breath sample transportation is also an issue with most conventional devices. The limited availability of instruments operable by patients and available at the point of care require samples to be shipped to central testing facilities, adding cost and inconvenience. A further difficulty arises in regard to a Urea Breath Test (UBT) from the high cost of ¹³C-urea, as well as the cost and operational expenses of instruments to detect exhaled ¹³CO₂. To solve this shortcoming, the present invention departs from detection of ¹³CO₂ by using unlabeled urea as a substrate, detecting ammonia in breath instead of CO₂, provides an ammonia-specific nanosensor and provides a simple, inexpensive hand-held device for the detection of breath NH₃.

Accordingly, a highly accurate medical device is provided that is economical, easy to operate, portable and sufficiently sensitive to diagnose medical conditions with a high degree of accuracy. The present invention provides a device and method for diagnostic analysis of exhaled/skin emission gases for reliable, low cost and non-invasive health care use.

### SUMMARY OF THE INVENTION

The present invention substantially solves the above shortcoming of conventional devices and provides at least the following advantages.

The present invention can avoid and reduces the need for serologic testing, for upper gastrointestinal endoscopy with mucosal biopsies, for H. pylori culture, including antimicrobial susceptibility testing, which is invasive and cumbersome, and for detection of H. pylori antigens in stool samples.

In a preferred embodiment, a medical device is provided to sample nasal air or gases emitted from a patient's skin to diagnose specific diseases, such as asthma, Chronic Obstructive Pulmonary Disease (COPD), cancer and metabolic disorders including high cholesterol and diabetes, via identification of disease-specific biomarkers.

An embodiment of the invention provides a medical device for analyzing gases in expired breath for facilitating diagnosis of a medical condition; the device includes sensing and gold substrates arranged on a TO8 substrate to provide highly reliable analysis. The sensing device is positioned to allow a gaseous sample to contact the sensing electrodes.

Another embodiment of the present invention provides a method for using the medical device of the present invention to analyze a patient's breath sample to diagnose the presence of a medical condition, by obtaining a breath sample from a patient; analyzing volatile components of the patient sample to provide a breath profile that includes both qualitative and quantitative data; comparing the patient's breath profile to a database of breath profiles, with each database profile being characteristic of at least one medical condition, to provide information pertinent to diagnosis of the presence or absence of a medical condition.

In a preferred embodiment, multiple tests performed on a single sample may be independent, or may be the results of several tests combined to produce a template or pattern representative of a patient's condition or representative of the presence of a particular compound or set of compounds. The high sensitivity of the nanomorphs of metal oxides prepared by sol-gel practices used in the medical device of the present invention are both more selective and more quantitatively precise than similar information obtained by currently available electronic nose technology. As a result, correlating the data pattern or changes in the data pattern over time identifies a wider range of conditions or compounds.

The present invention departs from the detection of ¹³CO₂ and provides a simplified assay that uses unlabeled urea as a substrate and detect ammonia in breath instead of CO₂, to provide specific nanosensors that detect breath ammonia or other breath components using a simple, portable and inexpensive hand-held device.

In preferred embodiments, the invention utilizes arrays of biocomposite and bio-doped films to provide a low cost, portable analyzer for detection of chemical products of biochemical reactions, such as ammonia and NO, in a real-time manner.

### DETAILED DESCRIPTION OF THE FIGURES

The above and other objects, features and advantages of certain exemplary embodiments of the present invention will be more apparent from the following detailed description taken in conjunction with the accompanying drawings, in which:

FIG. 1 is a schematic representation of an embodiment of the present invention;

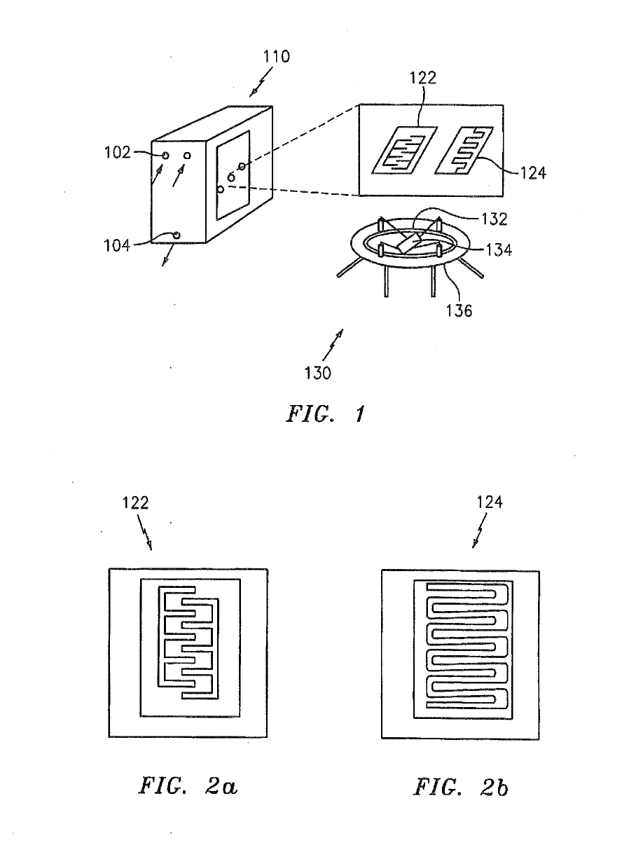

FIGS. 2a and 2b show heater and sensing electrodes of an embodiment of the present invention;

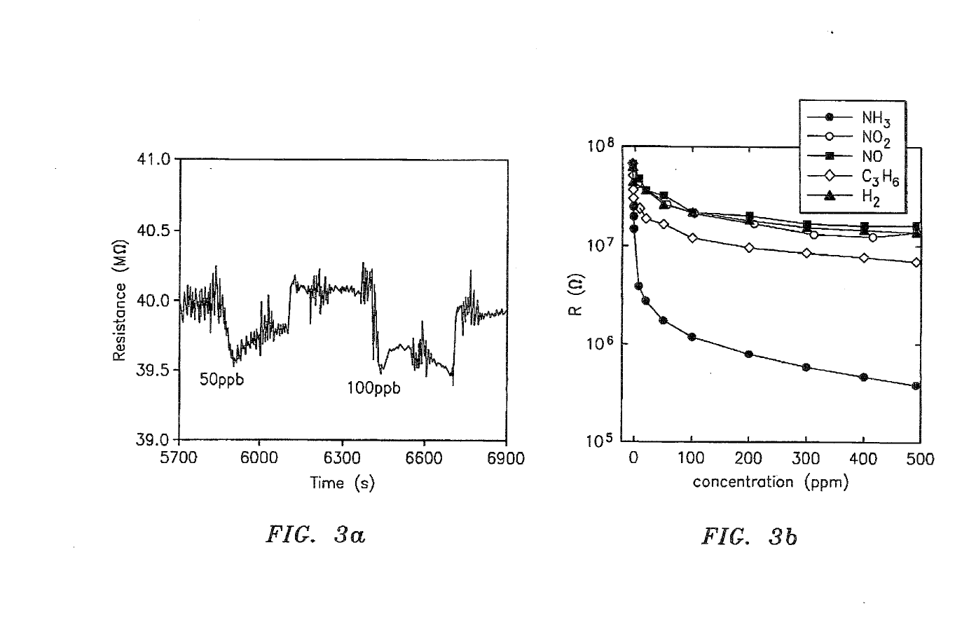

FIGS. 3a and 3b show sensor response;

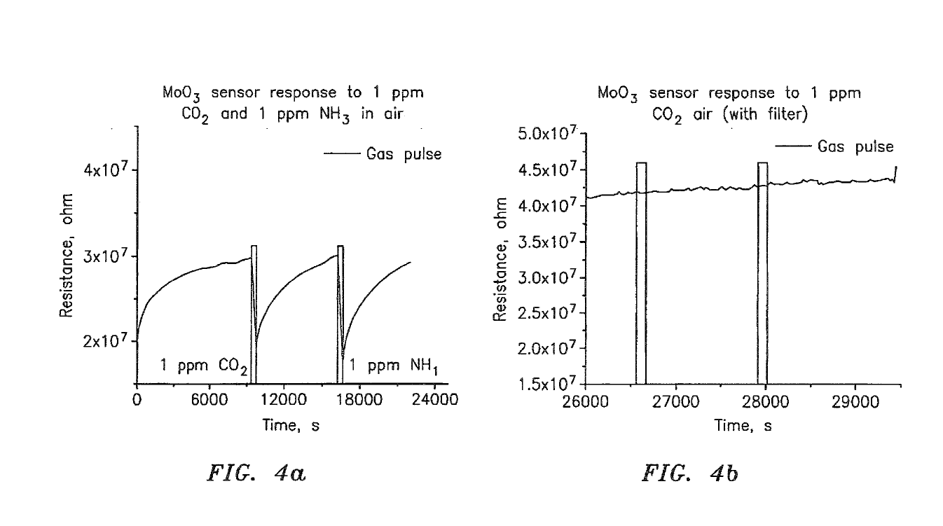

FIGS. 4a and 4b show NH₃ sensing and sensor response when exposed only to CO₂;

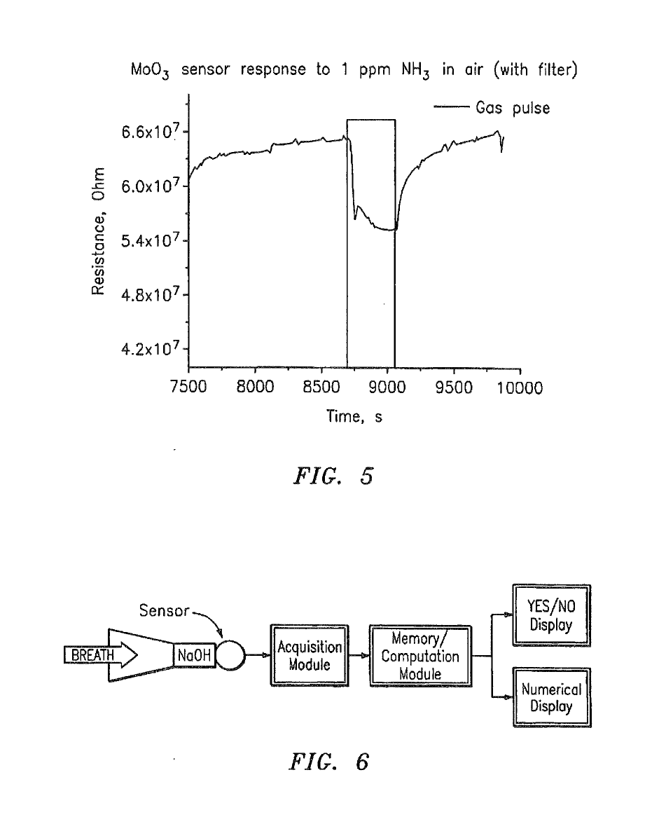

FIG. 5 shows NH₃ sensing with a CO₂ filter; and

FIG. 6 provides a block diagram of an apparatus of an embodiment of the present invention.

### DESCRIPTION OF THE PREFERRED EMBODIMENTS

The below description of detailed construction of preferred embodiments provides a comprehensive understanding of exemplary embodiments of the invention. Accordingly, those of ordinary skill in the art will recognize that various changes and modifications of the embodiments described herein can be made without departing from the scope and spirit of the invention. Descriptions of well-known functions and constructions are omitted for clarity and conciseness.

Analysis of breath and skin emission samples for diagnostic purposes has the advantage that the sample to be analyzed is collected from the patient in a non-invasive manner with a minimum of discomfort or inconvenience. Basic components of the medical device used for analysis in accordance with a preferred embodiment of the present invention are shown in FIG. 1. In preferred embodiments of the invention, breath samples are quantitatively and qualitatively processed. Notably, the sensor is tuned to detect NH₃ levels lower than 50 parts per billion (<50 ppb) and as high as 500 ppm, thereby covering all NH₃ levels encountered in humans, and in particular in patients undergoing UBT. Quantitative analyzers preferably include a sensing substrate surrounded by a gold substrate surrounded by a TO8 substrate. The medical to device of the present invention is preferably qualitatively used, to test for presence of an exhaled gas an/or gas emitted from a person's skin.

Qualitative tests performed by the present invention fall into two general types. First, the presence of the breath component alone may be significant to the health of the patient. This is particularly important where the chronic monitoring of the breath components of the patient indicate the absence of a component and that component appears in a new breath sample analysis. The converse change may also be significant, that is, a component formerly present is absent in the new breath sample analysis. A device in accordance with the present invention detects both conditions if maintenance of a patient's specific data history is desired and preserved in memory.

It can be significant that a newly detected component falls within a given range and the qualitative assessment of this newly detected component can be obtained using the medical device of the present invention. This is important where it is necessary to alert an attending physician whether the course of treatment, e.g. diet control, either for weight loss or for diabetes is actually working as desired. In accordance with embodiments of the present invention, data from a particular patient is stored so that multiple samples over an extended period of time may be taken. This permits a baseline to be established for a particular patient, and trend analysis is performed on the resulting data, relative to the database of spectroscopic breath profiles. If there is an acute and significant change in the chronic condition of the patient's breath, indications of this change may be communicated to a physician or healthcare provider via communications components linked to the computer.

The types of tests that may be employed include carbon dioxide content, alcohol content, lipid degradation products, aromatic compounds, thio compounds, ammonia and amines or halogenated compounds. As an example of the usefulness of detecting these components, lipid degradation products such as breath acetone are useful in monitoring diabetes. Compounds such as methanethiol, ethanethiol, or dimethyl sulfides have diagnostic significance in detecting widely differing conditions, such as psoriasis and ovulation. Increased ammonia has been associated with hepatic disease, although the present invention is not limited to detection of ammonia levels. Halogenated compounds may be indicative of environmental or industrial pollutants.

A baseline or breath composition history for a particular patient may also be compiled using the present invention. In this embodiment, an initialization test is first run on a sample of the patient's exhaled breath, with additional samples analyzed thereafter. As additional samples are analyzed and stored in memory at specific times over an extended period of time, the last stored or baseline sample data is then recalled from memory and the change or delta information between the new sample data and stored sample data is determined.

In a preferred embodiment, multiple different tests performed on a single sample may be independent, or may be the result of several tests combined to produce a template or pattern representative of a patient's condition or representative of the presence of a particular compound or set of compounds. The high sensitivity of the nanomorphs of metal oxides prepared by sol-gel practices used in the medical device of the present invention are both more selective and more quantitatively precise than similar information obtained by currently available electronic nose technology. As a result, correlating the data pattern or changes in the data pattern over time identifies a wider range of conditions or compounds.

The present invention departs from detection of ¹³CO₂ and provides a simplified assay that uses unlabeled urea as a substrate and detect ammonia in breath instead of CO₂ utilizing Equation (1):

CO(NH₂)₂+HOH—urease→CO₂+2NH₃ (1)

In an embodiment of the present invention, a nanosensor is provided to detect breath ammonia and a simple, portable, inexpensive hand-held device is thereby provided to detect breath NH₃. The nanosensor is tuned according to the method described below for other breath gases, and the nanosensor is in a preferred embodiment provided as a plug-in component. The sensor is constructed of a metal oxide that is not crystalline, raising sensitivity to ammonia and other gases.

In FIG. 1, a gas sample, i.e. breath or skin emission, accesses analyzer 110 via entry and exit orifices 102 and 104. A stainless steel chamber preferably connects the orifices to avoid absorption/distortion. Sensing electrode 122 and heater electrode 124 are positioned within the analyzer 110. The sensing electrode 122 includes a sensor 130 having gold substrate 132, sensing substrate 134 and TO8 substrate 136. Heater and sensing electrodes 122 and 124 of an embodiment of the present invention are shown in FIGS. 2a and 2b. Those of skill in the art recognize use of the TO8 substrate. Hirata et al. in U.S. Pat. No. 5,252,292, the contents of which are incorporated by reference herein, disclose a type of ammonia sensor.

In the present invention, the sensing electrode 124 is selectively tuned by spin or drop coating of sensing substrates generating film of MoO₃. In a preferred embodiment, a gel-sol synthesis was employed to produce three-dimensional (3-D) networks of nanoparticles, with the sol-gel processing preparing a sol, gelating the sol and removing the solvent. Molybdenum trioxide (MoO3) was prepared by an alkoxide reaction with alcohol according to Equation (2):

Molydenum (VI) Isopropoxide+1-Butanol→Precursor(0.1M) (2)

The prepared sol was spin coated and drop coated onto sensing substrates generating thin films of MoO₃. The sensing substrates (3 mm×3 mm) were made of Al₂O₃ and were patterned with interdigitated Pt electrodes. Pt heater electrodes were embedded on the rear of the sensor. The amorphous films were then calcined at higher temperatures generating polymorphic form. Differential scanning calorimetry confirmed the phase transformation.

FIG. 3a shows sensor response to NH₃, with the sensor generating a clear and measurable response to two NH₃ concentrations, 50 and 100 ppb. The measured amounts of ppb, i.e. parts per billion, are much lower than amounts typically expected in human breath, allowing for more accurate and expedited measurement and results. FIG. 3b shows sensor response to various breath gases, and the specificity regarding same. Shown in FIG. 3b are NH₃, NO₂, NO, C₃H₆ and H₂, gases that potentially interfere with NH₃ determination.

FIG. 4a shows NH₃ sensing without a CO₂ filter, as gas-sensing properties of the nanosensor. As shown in FIGS. 4a-b, when the sensor was exposed to various concentrations of NH₃ gas in a background mixture of N₂ and O₂ simulating ambient air, NH₃ was detected easily, down to 50 ppb, and even lower concentrations.

In FIG. 4a, CO₂ and NH₃, each at 1 ppm, produce similar responses to gas pulses, shown as vertical lines in FIG. 4a. Sensor response when exposed only to CO₂ gas, in the presence of the CO₂ filter, is shown in FIG. 4b. The CO₂ filter completely eliminates CO₂ from the gas stream, abrogating the sensor response to it.

Sensor specificity, in regard to sensing of NH3, was evaluated by exposing the sensor to various gases typically encountered in human breath, including NO₂, NO, C₃H₆, and H₂, each up to 490 ppm. Conductivity changes were measured in dry N₂ with 10% O₂. At 440° C. the film was very sensitive to NH₃, with 490 ppm increasing the conductivity by approximately a factor of 70, approximately 17 times greater than the response to the other gases. The NH₃ response, however, was relatively unaffected by 100 ppm of NO₂, NO, C₃H₆, and H₂. X-ray photoelectron spectroscopy (XPS) showed that the increased conductivity in the presence of NH₃ was accompanied by a partial reduction of the surface MoO₃. The resistance of the films increased after extended time at elevated temperatures.

CO₂ is an important component of human breath, with concentration in expired breath reaching up to 5%. Under test conditions, CO₂ interfered with NH₃ sensing. To overcome this limitation, a commercially available CO₂ filter (NaOH premixed with Vermiculite (V-lite) used in a 10:1 ratio; Decarbite absorption tube, PW Perkins and Co) was used. Decarbite reacts only with highly acidic gases such as CO₂, H₂S, thus excluding the possibility of cross adsorption; and the latter was verified. Exposing the sensor to various concentrations of NH₃ and CO₂, in the presence of N₂ and O₂, indicated that the presence of CO₂ did not affect NH₃ sensing. This was found to be true even when the two gases were at equal concentrations ranging between 0.5 and 10 ppm. Representative results of the evaluation of CO₂ interference with the NH₃ assay are shown. It was noted that the NaOH Decarbite traps CO₂ more efficiently at high CO₂ concentrations, and the data shown in FIGS. 4a-b are from experiments with a low CO₂ concentration (1 ppm). FIG. 5 shows NH₃ sensing with a CO₂ filter. In FIG. 5, the sensor is exposed to NH₃ in the presence of the filter, with no interference of the measurement. Combining NH₃ and CO₂ generated similar results, with the filter eliminating the experimental 1 ppm of CO₂ in the gas stream. Even at low concentrations, interference by CO₂ is eliminated.

Operation of apparatus of the present invention is based on sensor response modifying electrical resistance. That is, the MoO3 sensor is prepared with properties required for its intended use, with lower limits of detection for NH₃ well below the NH₃ concentrations typically found in human breath and, of course, below the increased NH₃ levels of a positive UBT.

In a preferred embodiment, MoO₃ nanosensor determines parameters of human breath and potentially interfering substances, such as those generated by H. pylori are detected. FIG. 6 shows a prototype for sensing breath, having a sensor, acquisition module, memory/computation module and displays.

While this invention has been particularly shown and described with reference to preferred embodiments thereof, it will be understood by those skilled in the art that various changes in form and details may be made therein without departing from the scope of the invention encompassed by the appended claims.

What is claimed is:

1. An apparatus for analyzing gases in expired breath to assist in diagnosis of a medical condition, the apparatus comprising a nanosensor tuned to a specific concentration profile, to detect a particular gaseous analyte, wherein the presence and/or concentration of said gaseous analyte is indicative of a particular medical condition.

2. The apparatus of claim 1, wherein the nanosensor is constructed of a metal oxide that is not crystalline.

3. The apparatus of claim 1, wherein the nanosensor is an ammonia-specific or ammonia selective nanosensor.

4. The apparatus of claim 1, wherein a plurality of specifically tuned nanosensors can be removably inserted into the apparatus.

5. The apparatus of claim 4, wherein insertion of a second specifically tuned nanosensor allows the apparatus to detect NO gas, insertion of a third specifically tuned nanosensor allows the apparatus to detect another volatile compound present in human breath, and insertion of additional specifically tuned sensors allows the apparatus to detect additional volatile compounds present in human breath.

6. The apparatus of claim 5, wherein the apparatus is a hand-held device that detects NH₃ in expired breath.

7. The apparatus of claim 1, wherein after the subject being evaluated ingests unlabeled urea as a substrate, levels of ammonia are measured in expired breath samples, to establish or rule out a diagnosis of infection with helicobacter pylori.

8. The apparatus of claim 7, wherein the substrate is selectively tuned by spin or drop coating.

9. The apparatus of claim 1, wherein the sensor is tuned to detect NH₃ in expired breath and generates a clear and measurable response for NH₃ concentrations ranging between at least of 50 parts per billion (ppb) and 500 parts per million (ppm).

10. The apparatus of claim 1, wherein presence of a selected gaseous analyte changes electrical resistance of the nanosensor.

11. The apparatus of claim 1, wherein the nanosensor incorporates biomolecule receptors in active, gas sensitive matrices.

12. The apparatus of claim 1, further comprising a baseline database of prior breath emissions for a particular patient.

13. The apparatus of claim 1, wherein the sensing device identifies molecular compounds in expired breath, said molecular compounds including ammonia, nitric oxide, ketones, methane, ethane, butane, pentane, carbon dioxide, carbon monoxide, oxygen, sulfur dioxide, carbon disulfide, hydrogen sulfide, methyl mercaptan, skatole, indole, cadaverine, putrescine, isovaleric acid, trimethylamine, and halogens, isoprene, isoprotanes, prostaglandins and halogen compounds.

14. The apparatus of claim 1, further comprising a baseline database of breath profiles identified as medical condition indicators.

15. The apparatus of claim 1, further comprising a microprocessor capable of identifying a change from a baseline established for a particular patient.

16. A method for utilizing a concentration profile in a breath sample to assist in the diagnosis of a medical condition, the method comprising:
measuring the concentration profile of a particular gaseous analyte in said breath sample with a nanosensor comprising a sensing electrode containing unlabeled urea as a substrate tuned for measuring said particular gaseous analyte; and
comparing the detected amounts of said gaseous analyte to a baseline to assist in diagnosis of a medical condition.

17. The method of claim 15, wherein the sensing electrode is tuned to detect ammonia and/or NH₃ levels.

18. The method of claim 15, wherein volatile components of the breath sample are analyzed to provide a breath profile including both qualitative and quantitative data; said method further comprising:
comparing the breath profile to a database of breath profiles characteristic of a plurality of medical conditions, to provide information pertinent to diagnosis of the presence or absence of a medical condition.

19. A method for determining biomolecule abundance, the method comprising:
obtaining a breath sample having a plurality of biomolecules; and
analyzing the sample utilizing an encapsulated sensor to determine whether a specific biomolecule in the sample matches a concentration profile of an analyte to which a nanosensor of the sensor tuned.

20. The method of claim 20, wherein the sensor can detect the specific biomolecule at a level of parts per billion.
